(19) United States
(12) Patent Application Publication
(10) Pub. No.: US 2024/0148238 A1
(43) Pub. Date: May 9, 2024

(54) SINGLE PIECE ARTICULATING JOINT

(71) Applicant: Boston Scientific Scimed, Inc., Maple Grove, MN (US)

(72) Inventor: Kian S. LIM, Shrewsbury, MA (US)

(73) Assignee: Boston Scientific Scimed, Inc., Maple Grove, MN (US)

(21) Appl. No.: 18/497,095

(22) Filed: Oct. 30, 2023

Related U.S. Application Data
(60) Provisional application No. 63/382,249, filed on Nov. 3, 2022.

(51) Int. Cl. A61B 1/008; A61B 1/00; A61B 1/005; A61L 29/02

(52) U.S. Cl. CPC A61B 1/008; A61B 1/0011; A61B 1/0055; A61L 29/02; A61M 25/0138

(57) ABSTRACT

An articulating joint for an endoscopic device includes a body and a wire. The body includes a first link at a proximal end, a last link at a distal end, and interior links therebetween. The body is formed as a tube with gaps. Each interior link is connected on a proximal end to a further proximal link via first flexible members and on a distal end to a further distal link via second flexible members. Each interior link forms a proximal gap between the interior link and the further proximal link, a distal gap between the interior link and the further distal link, and a recess on both sides of the members permitting the members to bend and the interior links to rotate. The wire extends through the links such that applying tension to the wire causes at least one link to rotate toward another link and articulate the joint.

### CROSS-REFERENCE TO RELATED APPLICATIONS

This application claims the benefit of priority to U.S. Provisional Application No. 63/382,249, filed on Nov. 3, 2022, which is incorporated by reference herein in its entirety.

### TECHNICAL FIELD

The present disclosure relates to an articulating joint for an endoscopic device that includes multiple links formed as a single piece.

### BACKGROUND

A minimally invasive medical device, such as an endoscope, may include a flexible elongate shaft comprising one or more channels through which a medical instrument can be run and/or controlled. Generally, an operating physician guides the endoscope to a target site within a living body (e.g., by passing the endoscope through a body lumen accessed, for example, via a body orifice) using an endoscopic camera or other guidance means so that a medical instrument may be used at the target site. In some endoscopes, the shaft includes an articulating joint (e.g., at a location along the shaft and/or at a distal end of the endoscope) to allow the operator to bend the endoscope through a desired amount of deflection in a desired direction. Known articulating joints often comprise a plurality of links connected by one or more pull wires (or other control elements) that can be tensioned or relaxed by the operator to control a curvature of the articulating joint and the flexible endoscope in which the articulating joint is placed.

The manufacture of endoscopes with such articulating joints may require difficult and/or time-consuming manual assembly processes in which each of the multiple links is added to a pull wire. If a link is positioned/oriented incorrectly, all of the previously assembled links may need to be removed and the process restarted, which can significantly increase labor costs.

### SUMMARY

The present disclosure relates to an articulating joint for an endoscopic device; the joint includes a body and at least one pull wire. The body extends longitudinally from a proximal end to a distal end and shaped into a plurality of links including a first link at the proximal end, a last link at the distal end, and a plurality of interior links between the first link and the last link. The body is formed as a single tube with gaps formed in the tube. Each interior link is connected on a proximal end to a further proximal link or the first link via first flexible members extending longitudinally between the interior link and the further proximal link and connected on a distal end to a further distal link or the last link via second flexible members extending longitudinally between the interior link and the further distal link. Each interior link is shaped so that a proximal gap is formed between the interior link and the further proximal link to permit the interior link to rotate toward the further proximal link or vice versa and a distal gap is formed between the interior link and the further distal link to permit the further distal link to rotate toward the interior link or vice versa, and a recess is formed on both sides of each of the flexible members to permit the flexible members to bend and permit rotation between the interior links about a transverse axis. The at least one pull wire extends longitudinally through the plurality of links from the proximal end to the distal end. Applying tension to the pull wire causes at least one link to rotate toward another link about the transverse axis and articulate the articulating joint.

In an embodiment, progressively applying further tension to the pull wire causes further links to rotate and further articulate the articulating joint into a desired shape or to a desired curvature.

In an embodiment, a size and shape of the links and a size and shape of the flexible members are selected to permit bending of the flexible members up to a maximum curvature when tension is applied to the pull wire.

In an embodiment, the maximum curvature of the flexible members is selected so that elastic deformation is maintained during articulation.

In an embodiment, a thickness of the tube is selected to permit the bending of the flexible members.

In an embodiment, a size and shape of the gaps and a size and shape of the recesses are selected so that a given link is able to rotate only until the link contacts an adjacent proximal link and the maximum curvature is achieved, whereupon the adjacent proximal link can rotate until it contacts a further adjacent proximal link.

In an embodiment, the proximal and distal gaps and the recesses are formed by laser cutting the tube.

In an embodiment, the tube is formed of stainless-steel.

In an embodiment, the tube is annealed before or after the laser cutting to increase the flexibility of the flexible members.

In an embodiment, the interior links include further gaps on a circumference of the link forming a crimp.

In an embodiment, a profile of the links and the flexible members are selected based on at least one of a desired bending radius, an articulating force, an articulating angle, and a coplanarity of the articulating joint.

In an embodiment, a profile of the links and the flexible members are selected so that cyclic stresses imposed during articulation of the articulating joint are insufficient to cause failure of the articulating joint.

In an embodiment, the articulating joint further includes lumens extending through the links offset from a longitudinal axis of the links, wherein the pull wire is run through the lumens so that applying tension to the pull wire applies a bending force to the links.

In an embodiment, the body is formed from Nitinol, plastic, or a polymer and from a micro-molding, 3D printing, or extrusion process.

In an embodiment, a profile of the links and the flexible members are selected so that articulation causes the distal end of the articulating joint to rotate 180 degrees into a cane shape; rotate 270 degrees into a P shape; or rotate 360 degrees into an O shape.

In addition, the present disclosure relates to a method for an endoscopic procedure. The method includes guiding an endoscopic device to a target site, the endoscopic device including an articulating joint comprising a body extending longitudinally from a proximal end to a distal end and shaped into a plurality of links including a first link at the proximal end, a last link at the distal end, and a plurality of interior links between the first link and the last link, wherein the body is formed as a single tube with gaps formed in the tube, wherein each interior link is connected on a proximal end to a further proximal link or the first link via first flexible members extending longitudinally between the interior link and the further proximal link and connected on a distal end to a further distal link or the last link via second flexible members extending longitudinally between the interior link and the further distal link, wherein each interior link is shaped so that a proximal gap is formed between the interior link and the further proximal link to permit the interior link to rotate toward the further proximal link or vice versa and a distal gap is formed between the interior link and the further distal link to permit the further distal link to rotate toward the interior link or vice versa, and a recess is formed on both sides of each of the flexible members to permit the flexible members to bend and permit rotation between the interior links about a transverse axis, the endoscopic device further including at least one pull wire extending longitudinally through the plurality of links from the proximal end to the distal end; and applying tension to the pull wire to cause at least one link to rotate toward another link about the transverse axis and articulate the articulating joint.

In an embodiment, progressively applying further tension to the pull wire causes further links to rotate and further articulate the articulating joint into a desired shape or to a desired curvature.

In an embodiment, a size and shape of the links and a size and shape of the flexible members are selected to permit bending of the flexible members up to a maximum curvature when tension is applied to the pull wire.

In an embodiment, a size and shape of the gaps and a size and shape of the recesses are selected so that a given link is able to rotate only until the link contacts an adjacent proximal link and the maximum curvature is achieved, whereupon the adjacent proximal link can rotate until it contacts a further adjacent proximal link.

In an embodiment, the tube is formed of stainless-steel and the proximal and distal gaps and the recesses are formed by laser cutting the tube.

Furthermore, the present disclosure relates to an articulating joint for an endoscopic device. The joint includes a body and at least one pull wire. The body extends longitudinally from a proximal end to a distal end and shaped into a plurality of links including a first link at the proximal end, a last link at the distal end, and a plurality of interior links between the first link and the last link, wherein each interior link is connected on a proximal end to a further proximal link via at least a first flexible member extending longitudinally between the interior link and the further proximal link and connected on a distal end to a further distal link via at least a second flexible member extending longitudinally between the interior link and the further distal link. The body is formed as a single piece. Each interior link is shaped so that a proximal gap is formed between the interior link and the further proximal link to permit the interior link to rotate toward the further proximal link or vice versa and a distal gap is formed between the interior link and the further distal link to permit the further distal link to rotate toward the interior link or vice versa, and a recess is formed on both sides of the first and second flexible members to permit the flexible members to bend and permit rotation between the interior links about a transverse axis in at least one direction. The at least one pull wire extends longitudinally through the plurality of links from the proximal end to the distal end. Applying tension to the pull wire causes at least one link to rotate toward another link about the transverse axis and articulate the articulating joint.

In an embodiment, progressively applying further tension to the pull wire causes further links to rotate and further articulate the articulating joint into a desired shape or to a desired curvature.

In an embodiment, a size and shape of the links and a size and shape of the flexible members are selected to permit bending of the flexible members up to a maximum curvature when tension is applied to the pull wire.

In an embodiment, the maximum curvature of the flexible members is selected so that elastic deformation is maintained during articulation.

In an embodiment, a thickness of the tube is selected to permit the bending of the flexible members.

In an embodiment, a size and shape of the gaps and a size and shape of the recesses are selected so that a given link is able to rotate only until the link contacts an adjacent proximal link and the maximum curvature is achieved, whereupon the adjacent proximal link can rotate until it contacts a further adjacent proximal link.

In an embodiment, the proximal and distal gaps and the recesses are formed by laser cutting.

In an embodiment, the body is formed of stainless-steel, Nitinol, or a polymer.

In an embodiment, the body is manufactured by micro-molding or 3D printing.

In an embodiment, a profile of the links and the flexible members are selected based on at least one of a desired bending radius, an articulating force, an articulating angle, and a coplanarity of the articulating joint.

### BRIEF DESCRIPTION OF THE DRAWINGS

FIG. 1 shows an articulating joint for use with an endoscopic device, the articulating joint comprising multiple links formed as a single piece, according to a first exemplary embodiment.

FIG. 2 shows a view of the proximal end of the articulating joint of FIG. 1.

FIG. 3 shows a portion of the articulating joint of FIG. 1 in greater detail, including a plurality of links with flexible members joining adjacent links.

FIG. 4 shows a cross section of the articulating joint of FIG. 1 including two lumens on opposing sides of the inner diameter of the joint through which respective pull wires can be run.

FIGS. 5-6 show a link of the articulating joint of FIG. 1 rotating toward an adjacent link proximal to the link.

FIG. 7 shows an articulating joint for use with an endoscopic device, the articulating joint comprising multiple links formed as a single piece, according to a second exemplary embodiment.

FIG. 8 shows a portion of the articulating joint of FIG. 2 in greater detail, including a plurality of links with flexible members joining adjacent links.

FIG. 9 shows a cross section of the articulating joint of FIG. 2 including two lumens on opposing sides of the inner diameter of the joint through which respective pull wires can be run.

FIGS. 10-11 show a link of the articulating joint of FIG. 2 rotating toward an adjacent link proximal to the link.

FIG. 12 shows the articulating joint of FIG. 7 in a fully articulated shape wherein each link is rotated to a maximum amount toward its adjacent link.

FIG. 13 shows a profile for a link of an articulating joint according to third exemplary embodiment.

### DETAILED DESCRIPTION

The present disclosure may be further understood with reference to the following description and the appended drawings, wherein like elements are referred to with the same reference numerals. The present disclosure relates to an articulating joint comprising multiple links for use with an endoscopic device. Those skilled in the art will understand that the multiple links may be formed from a single piece of material so that the articulating joint may be formed as a unitary member. The links are shaped or cut into a profile that permits thin, flexible members (or hinges) between adjacent links to bend and rotate the links relative to one another in one or more directions to curve the articulating joint. Each link further includes one or more lumens permitting one or more pull wires to extend longitudinally through the links to apply tension to rotate the links and curve the joint. The exemplary articulating joints described herein may be suitable for single use devices or for reusable devices, as will be described in further detail below.

The profile of each link may be designed, for example, based on desired specifications of the articulating joint, which may vary across different endoscopes or types of endoscopes. For example, the articulating joint may have a desired bending radius, articulating force, maximum articulating angle, coplanarity, etc. The links, and the flexible members connecting adjacent links, can be designed so that a gap exists between adjacent links with a size of each gap being selected to permit the adjacent links to deflect (e.g., rotate) through a desired range relative to one another when an articulating force is applied.

The rotation of the links in the disclosed embodiments is permitted by the bending of flexible members that connect the adjacent links to one another. The flexible members are made sufficiently thin/flexible such that a rotational force applied at an end of the flexible member bends the flexible member in a permitted direction. The bending of the flexible members is permitted by a recess on one or more sides of the flexible member into which the member can bend or curve before the adjacent links contact one another.

The articulating joint can include any number of links depending on the above design considerations and the specifications of the endoscope with which the articulating joint is to be used. In one exemplary embodiment, the articulating joint comprises 25 links and is sized/shaped for use with a LithoVue® ureteroscope. In another exemplary embodiment, the articulating joint comprises 20 links. However, those skilled in the art will ascertain that the number of links and the particular link profile can be selected based on any number of considerations. Additionally, the exemplary embodiments are described with respect to a link profile that permits rotation (e.g., bending or curving) in two directions via two different pull strings on opposing sides of the articulating joint via symmetrical link profiles mirrored across a longitudinal plane bisecting the articulating joint. However, the articulating joint can also be designed to permit rotation in a single direction or, potentially, more than two directions.

The exemplary embodiments are further described with respect to a link profile that is common to most or all of the links in the articulating joint. Using a common link profile (for at least the interior links) may simplify manufacturing techniques and provide uniformity to the curvature of the articulating joint. However, those skilled in the art will ascertain that different links in the same articulating joint can comprise different profiles, if desired to achieve a design objective or for any other reason.

In some exemplary embodiments, the first (most proximal) link or the last (most distal) link in the joint may have a profile different from the interior links between the first and last links, each of which, in an exemplary embodiment, has a common profile. In other embodiments, some interior links can comprise a profile different from other interior links to, e.g., permit the articulating joint to have different degrees of curvature in different portions of the joint.

The articulating joints according to the disclosed embodiments may be formed using any of a variety of manufacturing techniques including, e.g., micro-molding, 3D printing, extrusion, and/or laser cutting. Those skilled in art the art will ascertain that the various types of link profiles encompassed by the present disclosure may be better suited for manufacturing via one technique relative to other techniques depending on the complexity of the link profile or other design considerations. The articulating joint can be formed from materials including, e.g., stainless-steel, Nitinol, plastics, polymers (e.g., PEBAX®, Polyurethane, Polyamide or PEEK), or other materials. The articulating joint can be formed from (e.g., extruded as) tubing.

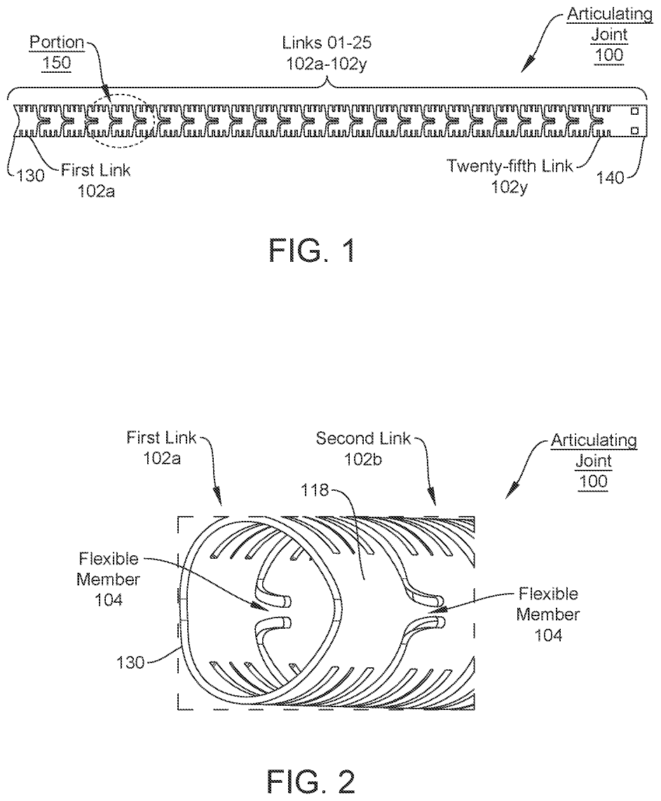

In one exemplary embodiment, e.g., an articulating joint 100 shown in FIGS. 1-6 may be manufactured by laser cutting a thin-walled stainless-steel piping to form the single stainless-steel pipe into multiple links interconnected via flexible members (hinges) as will be described in more detail below. In these aspects, the flexible members are designed so that the hinges deform only elastically during articulation of the joint. As would be understood by those skilled in the art, the area of and adjacent to the hinge that is affected by the heat from laser cutting may be minimized by employing picosecond or femtosecond laser cutting. The stainless-steel may also be annealed before or after laser cutting to increase the hinge flexibility.

FIGS. 1-6 show the articulating joint 100 for use with an endoscopic device, where the articulating joint 100 comprises multiple links 102 formed as an integrated entity formed from a single continuous piece of material, according to a first exemplary embodiment. In the example of FIGS. 1-6, the articulating joint 100 is formed from a tube (e.g., a stainless-steel tube) with a wall thickness t. The tube is laser cut to shape the links 102 and flexible members (hinges) 104 connecting adjacent links 102.

In this example, the articulating joint 100 includes 25 links, e.g., links 102a-102y shown in FIG. 1. The first link 102a is at a proximal end 130 of the articulating joint 100 and the twenty-fifth link 102y is at the distal end 140 of the articulating joint. The first link 102a of the articulating joint may couple to features at the proximal end of the endoscope, e.g., an extended flexible metal tube or polymer tubing, while the last link 102y may couple to features at the distal end of the endoscope, e.g., a distal cap or tip. With the exception of the first (most proximal) link 102a and the last (most distal) link 102y, each of the remaining (interior) links 102 (e.g., the second link 102b, the third link 102c, etc.) is attached to a further link 102 on a proximal side and a further link 102 on a distal side. Adjacent links 102 are connected to one another via two flexible members 104 on opposing sides of the circumference (i.e., diametrically opposed portions) of the links 102, as shown in FIG. 2. As will be described in more detail below, the articulating joint 100 is designed to flex in a plane perpendicular to the diameter connecting the two flexible members 104 of each link 102 to one another.

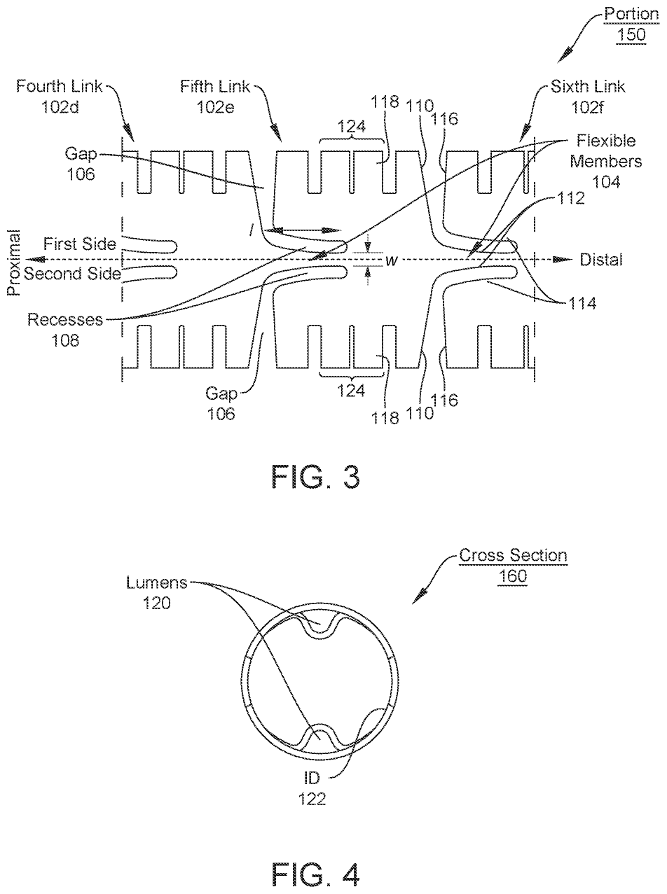

FIG. 3 shows a portion 150 of the articulating joint 100 of FIG. 1 in greater detail, including a plurality of links 102 with flexible members 104 joining adjacent links 102. Each of the interior links 102 in the present example (including e.g., the fourth link 102d, the fifth link 102e, and the sixth link 102f) comprises a section of tube. The tube from which the articulating joint 100 was formed has been laser cut to form gaps 106 around the circumference of the tube and recesses 108 on either side of the flexible members 104. It is noted that the size and shape of the gaps 106 and the recesses 108, defining the shape of the links 102, can vary, and these exemplary embodiments are not limited to the design shown in FIGS. 1-6. Although the gaps 106 and the recesses 108 are described as separate spaces, it should be understood that these spaces make up a single continuous space and the distinction between the two is merely provided for ease of explanation.

The surfaces defining the links 102 and the flexible members 104 are defined in part by the thickness t of the tube. The distal surfaces 110 of the link 102 extend around the circumference of the tube between the flexible members 104. The flexible members 104 are defined by a longitudinal length l and a width w (and a depth d equal to the thickness t). It should be understood that the width w has a slight curve corresponding to the curvature of the tube over a small circumferential distance. In the example shown in FIG. 3, the width w of the flexible member may not be uniform and one end of the flexible member may have an increased width w. In this example, the proximal end of the flexible member is flared so that the bending stress imposed during articulation is spread around a larger area to prevent plastic deformation and/or shearing of the flexible member. The size and shape of the flexible members 104 (defined by l, w and t) in combination with the properties of the material from which the flexible members are formed can be designed to prevent premature failure from the cyclic stresses imposed during articulation. If the articulating joint is designed for single-use, fewer cyclic stresses will be imposed than if the articulating joint is designed for a reusable device. Thus, the profile of the flexible members 104 may depend on the intended use of the articulating joint. These stresses can be modeled using a computer aided drafting (CAD) simulation platform and verified by actual testing of the articulating joint.

A first surface 112 (of the flexible member 104) and a second surface 114 (of the link 102 distal to the flexible member 104) define the recesses 108. The proximal surfaces 116 of the link 102 extend around the circumference of the tube between the recesses 108. The length of the link 102 is defined by the outer surface 118 of the section of tube from which the link 102 was formed, specifically, from the most proximal location of proximal surfaces 116 to the most distal location of the distal surfaces 110. Those skilled in the art will ascertain that the delineation between a first link, a flexible member, and a second link, and the definition of the various surfaces, are selected for ease of explanation.

FIG. 4 shows a cross section 160 of the articulating joint 100 of FIG. 1 including two lumens 120 on opposing sides of the inner diameter 122 of the articulating joint 100 through which respective pull wires can be run. The lumens 120 extend through the length of the articulating joint, e.g., through each of the links 102. Using a coordinate system where the X-axis is the longitudinal axis of the link 102, the Y-axis is in a first direction orthogonal to the X-axis (in this case, “up and down”), and the Z-axis is in a second direction orthogonal to both the X and Y axes (in this case, “left and right”), the longitudinal axes of the lumens 120 are parallel to and offset from the longitudinal axis of the link 102 in a first transverse direction (e.g., in Y direction) and the longitudinal axes of the flexible members 104 are offset from the longitudinal axis of the link 102 in a second transverse direction (e.g., in X direction) orthogonal to the first transverse direction.

In other words, assuming the positive Y axis as 0 deg on the circumference of the tubing, the lumens 120 are at 90 deg and 270 deg and the flexible members 104 are at 0 deg and 180 deg. The pull wire can be fixed on the most distal link 102y on the distal end 140 of the articulating joint 100 and free on the proximal end 130 so that an operating physician can apply tension to the pull wire. The tension provides an axial force in the proximal direction from the fix point of the wire, e.g., the most distal link 102y. In this arrangement, the articulating joint 100 will flex in a bending plane including the link 102y.

When tension is applied to the pull wire, a force is applied to the link 102y along the axis of the lumen 120 offset from the transverse axis of the link 102y between the two flexible members 104 connecting the most distal link 102y to the adjacent link 102x proximal to the most distal link 102y. This applies a bending force to the flexible members 104 so that the flexible members 104 bend in the direction of the pull wire, causing the most distal link 102y to rotate in the bending plane so that the distal link 102y is drawn laterally outward and, depending on the amount of tension applied, may be bent so that a distal end of the distal link 102y is drawn toward the adjacent link 102x.

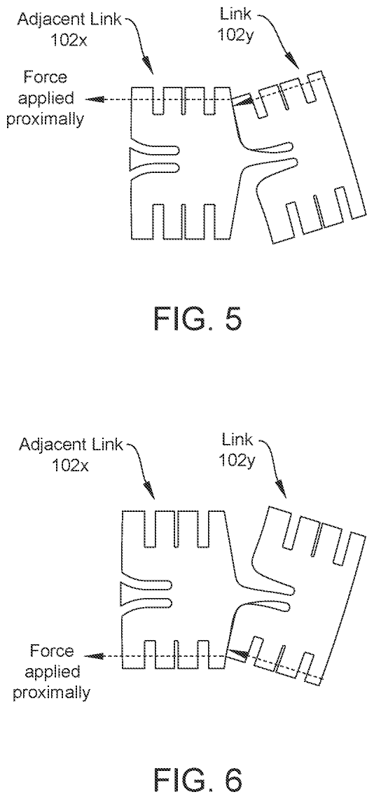

FIGS. 5-6 show a link 102y of the articulating joint 100 of FIG. 1 rotating toward an adjacent link 102x proximal to the link 102y. In FIG. 5, a proximal force is applied to a pull wire on a first side of the link 102y. The recess 108 allows the flexible member(s) 104 to bend. The flexible member 104 connecting the links 102 are bent toward the pull wire on the first side (i.e., the articulating joint 100 is bent so that the side of the articulating joint 100 along which extends the pull wire being drawn proximally, forms the inner diameter of the bent section of the articulating joint 100. The first side of the link 102y (e.g., defined by the proximal surface 116) is brought into the gap 106. The bending reaches its maximum when the proximal surface 116 of the link 102y is brought into contact with the distal surface 110 of the adjacent link 102x.

At the same time, bending proceeds in substantially the same manner and to substantially the same extent in each of the links 102 so that the articulating joint 100 takes on a curvature that is substantially equal along the length of the articulating joint 100. In FIG. 6, a proximal force is applied to a pull wire on a second side of the link 102y and the link 102y is rotated in the opposing direction. Thus, the articulating joint can be articulated into a desired shape (e.g., to a desired degree of curvature) or to a desired curvature based on the tension applied to the pull wire.

As described above, the profile of the links 102 and the flexible members 104 permits the flexible members 104 between adjacent links 102 to bend so that the links 102 may rotate relative to one another to provide a curve to the articulating joint 100. The profile of each link 102 can be designed based on the desired specifications of the articulating joint 100, which may vary across different endoscopes or types of endoscopes. For example, the articulating joint may have a desired bending radius, articulating force, maximum articulating angle, coplanarity, etc. The thickness t of the tube is selected to be sufficiently small so that the flexible member 104 can flex. The stainless-steel tube forming the articulating joint 100 can be annealed before or after laser cutting.

Additionally, in the example of FIGS. 1-6, the link 102 includes further gaps that form a crimp 124 in the link 102. The crimp 124 provides structure for the pull wire to pass through while retaining sufficient flexibility for these portions of tube to, e.g., deform or deflect slightly during articulation. The interval of crimping along the articulating joint 100 can allow the articulating joint 100 to bend into the desired shape, e.g., a “candy cane” shape, a “P” shape or an “O” shape.

As described above, in various other examples, the articulating joint can be formed by various manufacturing techniques including, e.g., micro-molding, 3D printing, extrusion, and/or laser cutting. Those skilled in art the art will ascertain that the various types of link profiles encompassed by the present disclosure may be better suited for manufacturing via one technique relative to other techniques depending on the complexity of the link profile or other design considerations. The articulating joint can be formed from materials including, e.g., stainless-steel, Nitinol, plastics, polymers, or other materials.

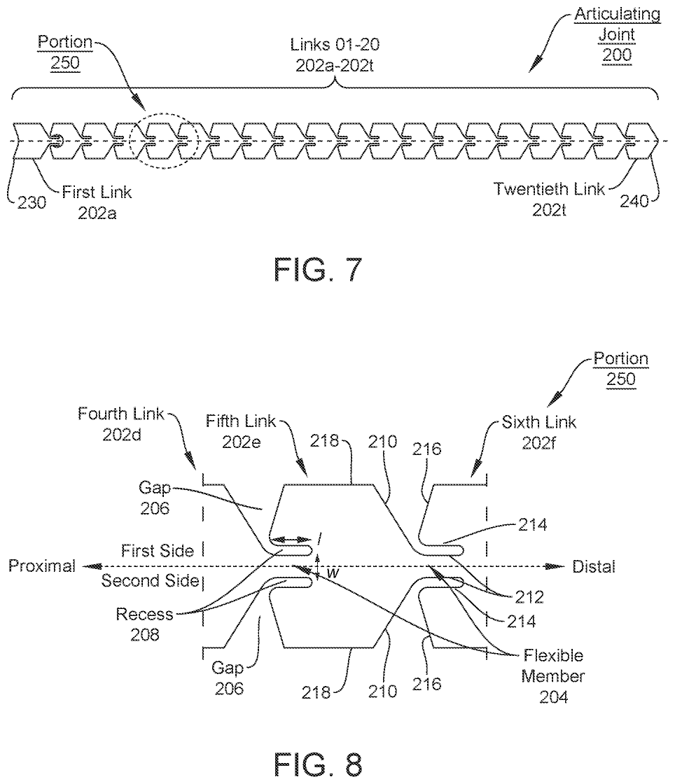

FIGS. 7-12 shows an articulating joint 200 for use with an endoscopic device, the articulating joint 200 comprising multiple links 202 formed as a single piece, according to a second exemplary embodiment. In the example of FIGS. 7-12, the articulating joint 200 is formed from a single, unitary tube. In this example, the articulating joint 200 includes 20 links, e.g., links 202a-202t, wherein the first link 202a is at a proximal end 230 of the articulating joint 200 and the twentieth link 202t is at a distal end 240 of the articulating joint 200. Each pair of adjacent links 202 is connected via two flexible members 204 on opposing sides of the circumference of the links 202. Relative to the articulating joint 100 described in FIGS. 1-6, the articulating joint 200 described in FIGS. 7-12 may be formed of material with greater flexibility than the stainless-steel of the articulating joint 100. Thus, the flexible members 204 may be thicker (in width and depth) and shorter (in length) than those described for the articulating joint 100. Additionally, the crimp 124 of the articulating joint 100 is not used in the articulating joint 200.

FIG. 8 shows a portion 250 of the articulating joint 200 of FIG. 2 in greater detail, including a plurality of links 202 with flexible members 204 joining adjacent links 202. Each of the interior links 202 in the present example (including e.g., the fourth link 202d, the fifth link 202e, and the sixth link 202f) comprises a section of tube. Similar to the articulating joint 100 of FIGS. 1-6, the articulating joint 200 is formed with gaps 206 extending around the circumference of the tube (except for the width of the flexible members 204) and recesses 208 on either side of the portion of the flexible members 204 adjacent to the proximal end of the more distal link of each adjacent pair of links 202. Although the gaps 206 and the recesses 208 are described as separate spaces, it should be understood that these spaces make up a single, continuous space and the distinction between the two is merely provided for ease of explanation.

The surfaces defining the links 202 and the flexible members 204 are defined in part by the thickness t of the tube. The distal surfaces 210 of each link 202 extend around the circumference of the tube between the flexible members 204. The flexible members 204 are defined by a longitudinal length l and a width w (as well as a depth d equal to the thickness t). Similar to the links 102 described in FIGS. 1-6, the width w has a slight curve corresponding to the curvature of the tube over a small circumferential distance.

A first surface 212 (of the flexible member 204) and a second surface 214 (of the link 202 distal to the flexible member 204) define the recesses 208. The proximal surfaces 216 of each link 202 extend around the circumference of the tube between the gaps 206. The length of each link 202 is defined by outer surface 218 of the section of tube from which the link 202 was formed, specifically, from the most proximal location of proximal surfaces 216 to the most distal location of the distal surfaces 210. Those skilled in the art will ascertain that the delineation between a first link, a flexible member, and a second link, and the definition of the various surfaces, are selected for ease of explanation. The proximal end of the flexible member 204 transitions into the distal surfaces 210 of the proximal link 202 in a flared shape, similar to the flexible members 104 and links 102 of the articulating joint 100 of FIGS. 1-6.

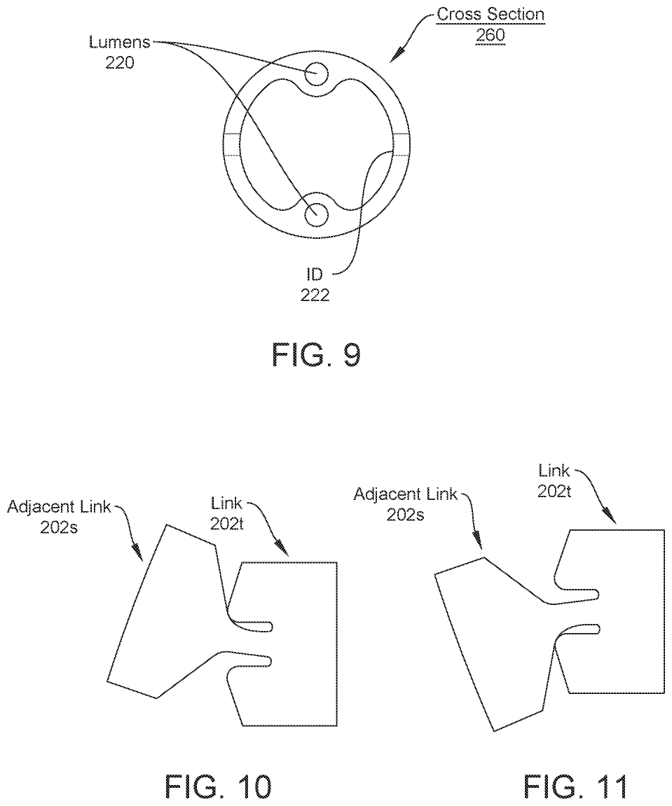

FIG. 9 shows a cross section 260 of the articulating joint 200 of FIG. 7 including two lumens 220 on opposing sides of the inner diameter 222 of the joint 200 through which respective pull wires can be run. The lumens 220 are positioned relative to the flexible members 204 so that tension can be applied to a pull wire running through the lumen 220 to provide an axial force in the proximal direction from the fix point of the wire, e.g., the most distal link 202t, similarly to the arrangement described for the articulating joint 100 of FIGS. 1-6. As with the articulating joint 100, the joint 200 is configured to bend in a plane including the lumens 220 of the various links 202.

FIGS. 10-11 show a link 202t of the articulating joint 200 of FIG. 7 rotating toward an adjacent link 202s proximal to the link 202t. In FIG. 10, a proximal force is applied to a pull wire on a first side of the link 202t. The recess 208 allows the flexible member(s) to bend. The flexible member 204 connecting the links 202 are bent toward the pull wire on the first side. The first side of the link 202t (e.g., defined by the proximal surface 216) is brought into the gap 206. The bending reaches its maximum when the proximal surface 216 of the link 202t is brought into contact with the distal surface 210 of the adjacent link 202s. In FIG. 11, a proximal force is applied to a pull wire on a second side of the link 202t and the link 202t is rotated in the opposing direction.

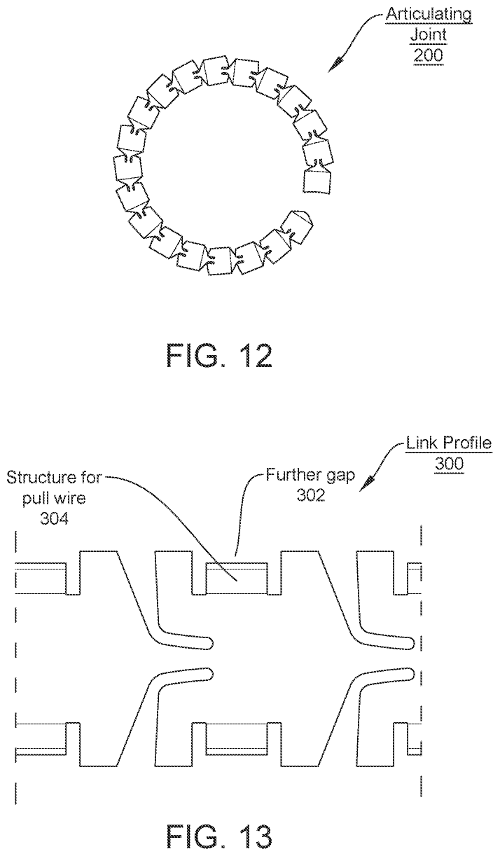

FIG. 12 shows the articulating joint 200 of FIG. 7 in a fully articulated shape wherein each link 202 is rotated to a maximum amount toward its adjacent link. As shown in FIG. 12, a full (or close to) 360 deg of curvature can be realized given a certain degree of curvature between adjacent links and a sufficient number of links that, when the angles between the articulated links are summed, the total rotation from the most proximal link 202a to the most distal link 202a reaches (or nearly reaches) 360 deg. This degree of articulation may be useful for articulating joints placed near the distal tip of the endoscope (e.g., not running the entire length of the endoscope), to provide fine control of the positioning of the distal tip.

FIG. 13 shows a profile for a link 300 of an articulating joint according to third exemplary embodiment. The profile for the link 300 is similar to that described for the links 102 of the articulating joint 100 of FIGS. 1-6. For example, the link 300 can be formed from pipe/tube (or shaped into a tube-like structure) and laser cut to form the links and flexible members. In this example, the outer surfaces of the links (e.g., the exterior of the tubing) are cut/formed so that further gaps 302 are made in the surface that permit further flexibility of the links 300. A structure 304 may be used to define the lumen for the pull wires to pass through.

As described above, the link profile can be designed based on any number of considerations to achieve a variety of design objectives for the articulating joint. Exemplary designs for articulating joints were provided above, however, the exemplary embodiments are not limited thereto. In other embodiments, the articulating joint may not comprise tubing and the forming of the various gaps, surfaces, and flexible members may not be limited to the thickness of a tube. For example, in other embodiments, the links may be formed as three-dimensional shapes. In these embodiments, a single flexible member extending substantially along the longitudinal axis of the link may join adjacent links. Those skilled the art will ascertain that any link design that provides sufficient gaps/recesses to permit the deflection of the links, according to the considerations described above, may be used.

It will be appreciated by those skilled in the art that changes may be made to the embodiments described above without departing from the inventive concept thereof. It should further be appreciated that structural features and methods associated with one of the embodiments can be incorporated into other embodiments. It is understood, therefore, that this invention is not limited to the particular embodiment disclosed, but rather modifications are also covered within the scope of the present invention as defined by the appended claims.

What is claimed is:

1. An articulating joint for an endoscopic device, comprising:
a body extending longitudinally from a proximal end to a distal end and shaped into a plurality of links including a first link at the proximal end, a last link at the distal end, and a plurality of interior links between the first link and the last link, wherein the body is formed as a single tube with gaps formed in the tube, wherein each interior link is connected on a proximal end to a further proximal link or the first link via first flexible members extending longitudinally between the interior link and the further proximal link and connected on a distal end to a further distal link or the last link via second flexible members extending longitudinally between the interior link and the further distal link,
wherein each interior link is shaped so that
a proximal gap is formed between the interior link and the further proximal link to permit the interior link to rotate toward the further proximal link or vice versa and a distal gap is formed between the interior link and the further distal link to permit the further distal link to rotate toward the interior link or vice versa, and
a recess is formed on both sides of each of the flexible members to permit the flexible members to bend and permit rotation between the interior links about a transverse axis; and
at least one pull wire extending longitudinally through the plurality of links from the proximal end to the distal end, wherein applying tension to the pull wire causes at least one link to rotate toward another link about the transverse axis and articulate the articulating joint.

2. The articulating joint of claim 1, wherein progressively applying further tension to the pull wire causes further links to rotate and further articulate the articulating joint into a desired shape or to a desired curvature.

3. The articulating joint of claim 1, wherein a size and a shape of the links and a size and a shape of the flexible members are selected to permit bending of the flexible members up to a maximum curvature when the tension is applied to the pull wire.

4. The articulating joint of claim 3, wherein the maximum curvature of the flexible members is selected so that elastic deformation is maintained during articulation.

5. The articulating joint of claim 3, wherein a thickness of the tube is selected to permit the bending of the flexible members.

6. The articulating joint of claim 3, wherein a size and a shape of the gaps and a size and a shape of the recesses are selected so that a given link is able to rotate only until the link contacts an adjacent proximal link and the maximum curvature is achieved, whereupon the adjacent proximal link can rotate until it contacts a further adjacent proximal link.

7. The articulating joint of claim 1, wherein the proximal and distal gaps and the recesses are formed by laser cutting the tube.

8. The articulating joint of claim 7, wherein the tube is formed of stainless-steel.

9. The articulating joint of claim 7, wherein the tube is annealed before or after the laser cutting to increase the flexibility of the flexible members.

10. The articulating joint of claim 1, wherein the interior links include further gaps on a circumference of the link forming a crimp.

11. The articulating joint of claim 1, wherein a profile of the links and the flexible members are selected based on at least one of a desired bending radius, an articulating force, an articulating angle, or a coplanarity of the articulating joint.

12. The articulating joint of claim 1, wherein a profile of the links and the flexible members are selected so that cyclic stresses imposed during articulation of the articulating joint are insufficient to cause failure of the articulating joint.

13. The articulating joint of claim 1, further comprising:
lumens extending through the links offset from a longitudinal axis of the links, wherein the pull wire is run through the lumens so that applying the tension to the pull wire applies a bending force to the links.

14. The articulating joint of claim 1, wherein the body is formed from one of Nitinol, a plastic, or a polymer and from one of a micro-molding, 3D printing, or an extrusion process.

15. The articulating joint of claim 1, wherein a profile of the links and the flexible members are selected so that articulation causes the distal end of the articulating joint to rotate 180 degrees into a cane shape, rotate 270 degrees into a P shape, or rotate 360 degrees into an O shape.

16. A method for an endoscopic procedure, comprising:
guiding an endoscopic device to a target site, the endoscopic device including an articulating joint comprising a body extending longitudinally from a proximal end to a distal end and shaped into a plurality of links including a first link at the proximal end, a last link at the distal end, and a plurality of interior links between the first link and the last link, wherein the body is formed as a single tube with gaps formed in the tube, wherein each interior link is connected on a proximal end to a further proximal link or the first link via first flexible members extending longitudinally between the interior link and the further proximal link and connected on a distal end to a further distal link or the last link via second flexible members extending longitudinally between the interior link and the further distal link, wherein each interior link is shaped so that a proximal gap is formed between the interior link and the further proximal link to permit the interior link to rotate toward the further proximal link or vice versa and a distal gap is formed between the interior link and the further distal link to permit the further distal link to rotate toward the interior link or vice versa, and a recess is formed on both sides of each of the flexible members to permit the flexible members to bend and permit rotation between the interior links about a transverse axis, the endoscopic device further including at least one pull wire extending longitudinally through the plurality of links from the proximal end to the distal end; and
applying tension to the pull wire to cause at least one link to rotate toward another link about the transverse axis and articulate the articulating joint.

17. The method of claim 16, wherein progressively applying further tension to the pull wire causes further links to rotate and further articulate the articulating joint into a desired shape or to a desired curvature.

18. The method of claim 16, wherein a size and a shape of the links and a size and a shape of the flexible members are selected to permit bending of the flexible members up to a maximum curvature when the tension is applied to the pull wire.

19. The method of claim 18, wherein a size and a shape of the gaps and a size and a shape of the recesses are selected so that a given link is able to rotate only until the link contacts an adjacent proximal link and the maximum curvature is achieved, whereupon the adjacent proximal link can rotate until it contacts a further adjacent proximal link.

20. The method of claim 19, wherein the tube is formed of stainless-steel and the proximal and distal gaps and the recesses are formed by laser cutting the tube.
